(19) United States
(12) Patent Application Publication
(10) Pub. No.: US 2013/0338382 A1
(43) Pub. Date: Dec. 19, 2013

(54) REACTION APPARATUS AND METHOD USING SUPERCRITICAL WATER OR SUBCRITICAL WATER

(71) Applicant: Hitachi, Ltd., Tokyo (JP)

(72) Inventors: Takeyuki KONDO, Hitachi (JP); Masayuki KAMIKAWA, Hitachinaka (JP); Toshiaki MATSUO, Mito (JP); Masashi TANTO, Tokyo (JP); Yasunari SASE, Tokyo (JP); Hiroyuki ITO, Tokyo (JP)

(21) Appl. No.: 13/907,060

(22) Filed: May 31, 2013

(30) Foreign Application Priority Data
May 31, 2012 (JP) 2012-124198

(51) Int. Cl. B01J 8/10; C13K 1/02; C07D 307/46

(52) U.S. Cl. CPC B01J 8/10; C07D 307/46; C13K 1/02
USPC 549/498; 422/162; 127/37

(57) ABSTRACT

In a method and apparatus for producing a useful substance by allowing a fluid containing a biomass raw material to act on a supercritical water and/or subcritical water, the fluid containing a biomass raw material in a high concentration is efficiently mixed with the supercritical water and/or subcritical water, whereby the amount of tar and carbon particles produced as by-products is decreased and blockage and abrasion of a pipe and an equipment is suppressed, or it is possible to easily remove the by-products.

At least two inlet flow paths for flowing the raw material fluid and the supercritical water or subcritical water into the mixing flow path are provided, along with an agitation blade having a rotating shaft set on a center shaft of the mixing flow path and an agitation blade having a rotating shaft set on a center shaft of the mixing flow path.

### BACKGROUND OF THE INVENTION

1. Field of the Invention

The present invention relates to a reaction apparatus and a method using a supercritical water or subcritical water. In particular, this invention relates to an apparatus and a method for allowing a supercritical water or subcritical water to act on a biomass raw material fluid to synthesize a beneficial chemical substance.

2. Description of Related Art

1,3-Propanediol is a raw material of high-quality polyester fibers inclusive of polytrimethylene terephthalate, and therefore, its demand is increasing in recent years. As one of synthesis methods of 1,3-propanediol, there is an acrolein hydration and hydrogenation method described in Tetsuya Harada, “Production, application and economic efficiency of 1,3-PDO and PTT”, CMC Publishing Co., Ltd., Planet Division, August 2000. This method is concerned with the production of 1,3-propanediol in which acrolein obtained by oxidizing propylene that is a crude oil material with air in the presence of a catalyst is subjected to a hydration and hydrogenation reaction, and is established as an industrial production method. However, because of a remarkable rise of the crude oil price in recent years, the development of a synthesis method from a biomass raw material is desired.

Though there has not been reported a method for chemically synthesizing 1,3-propanediol from a biomass raw material, technologies for synthesizing acrolein that is a precursor exist, and one of the technologies is described in Masaharu Watanabe, et al., “Acrolein synthesis from glycerol in hot-compressed water”. This method is a method in which a glycerin aqueous solution that is a biomass raw material is mixed with a high-temperature supercritical water at 35 MPa using a small-scale apparatus having a pipe diameter in the order of 1 mm and a flow rate of from 10 to 50 mL/min, and the temperature is instantly raised to 400° C., thereby synthesizing acrolein. Here, an optimum reaction time is about 20 seconds. This method has a characteristic feature in the point that a proton originating from the addition of a trace amount of sulfuric acid to the glycerin aqueous solution functions as a catalyst for accelerating a dehydration reaction of glycerin.

However, according to the method of Masaharu Watanabe, et al., “Acrolein synthesis from glycerol in hot-compressed water”, the concentration of glycerin in the raw material is low as about 1%, and much energy is consumed for raising the temperature and pressure of water. Accordingly, for the production on a commercial basis, it is necessary to increase the concentration of glycerin in a reaction solution to an extent of at least 15% or more. If the concentration of glycerin is increased to 15% or more, the optimum reaction time becomes short as several seconds due to an increase of the reaction rate. For this reason, it is at least necessary to achieve complete mixing for a time of 1/10 of the optimum reaction time.

However, since a difference in the viscosity between the supercritical water and the glycerin aqueous solution also increases with an increase of the concentration of glycerin, the mixing properties are lowered. Furthermore, in a commercial plant on a scale of several tons per year, in the case of mixing the reaction solution at an economical flow rate, the pipe diameter becomes a size of from 1 to 10 cm, and following this, a diffusion distance also increases. At that time, since the mixing time is in inverse proportion to a square of the pipe diameter, the mixing time becomes several seconds or more. In the case where the mixing properties are lowered, the coordination number of supercritical water in the vicinity of a glycerin molecule is lowered.

FIG. 1 shows a dehydration reaction route of glycerin using a supercritical water. When the coordination number becomes low, a side reaction dominantly proceeds rather than a main reaction for forming acrolein, and therefore, a reaction yield of acrolein is lowered. In addition, since glycerin comes into contact with a supercritical water to cause a reaction at a temperature higher than the reaction temperature with a lowering of the mixing properties, the amount of reaction by-products produced, such as tar and carbon particles, increases, and furthermore, the yield is lowered.

Then, the carbon particles coagulated by the tar adhere to a valve element and a valve seat of a valve. According to this, for example, the valve element and the valve seat are abraded, and an operation range of the valve element is restricted. Thus, there may be a possibility that it is difficult to precisely control the pressure. In addition, when the tar produced deposits onto the reaction pipe wall surface, the wall surface is carbonized, and therefore, blockage of the pipe is liable to occur. Then, from the viewpoints of an increase of the concentration of glycerin and scaling up, it is necessary to improve the mixing properties and to remove the tar deposited onto the reaction pipe wall surface.

In a method for treating an organic waste water with a supercritical water, a technology for preventing the deposition of a salt onto the reaction pipe wall surface is disclosed in JP-A-9-299966. In general, while water in a state at ordinary temperature and normal pressure has large dielectric constant and high solubility of salts, water in a supercritical state is liable to cause deposition of salts due to a decrease of the dielectric constant. As shown in FIG. 2, the technology disclosed in JP-A-9-299966 is concerned with a system including a reaction vessel configured of a double-pipe structure wherein an inner pipe thereof is composed of a porous cylinder, in which an organic waste water, a supercritical water, and a neutralizing agent of reaction solution (alkaline aqueous solution) are sent to the inside of the porous cylinder to decompose an organic material, and air is discharged from the outside of the porous cylinder toward the inside, thereby suppressing deposition onto the wall surface by a salt solid produced due to neutralization of the reaction solution. However, there is involved such a problem that since the inner wall of a reaction pipe is porous, when the salt once deposits, it enters the inside of the pore and cannot be removed.

In a method for treating an organic waste water with a supercritical water, a technology for removing a salt deposited onto the reaction vessel wall surface is disclosed in JP-A-10-015566. As shown in FIG. 3, this method is a method in which an organic material, an oxidizing agent, and a supercritical water are fed into a vertical cylindrical reaction vessel, and a salt deposited onto the reaction vessel wall surface at the time of decomposing the organic material is scraped off by moving a scraper vertically. The scraper is always positioned within a subcritical region, and at the time of scraping-off, the scraper is transferred into a supercritical region and moved vertically. Thus, the salt substance scraped off is dissolved in a subcritical water region, and therefore, there is such an advantage that a problem of deposition and accumulation of the salt substance on the scraper itself is scarce. However, though the salt can be dissolved, carbon does not have solubility against water in any state, and the scraper is moved vertically in high-pressure water. Thus, there is involved such a problem that much energy is required at the time of pushing the scraper.

In addition, a method for removing a deposit on the pipe wall surface in an outer pipe of a self-heat recovery type double-pipe heat exchanger is disclosed in JP-A-11-114600. As shown in FIG. 4, this method is a method in which a magnet-made annular scraper is set in the inside of the outer pipe of the double-pipe heat exchanger, and by transferring this by a magnet set in the outside, a solid deposited onto the pipe wall surface is scraped off and removed. However, there is involved such a problem that the solid deposited onto the inner surface of an inner pipe of the double-pipe heat exchanger or on the wall surface of the reaction pipe which will become high in the temperature cannot be removed.

### SUMMARY OF THE INVENTION

An object of this invention is to provide a technology regarding a method for commercially producing a useful substance at a large flow rate by allowing a fluid containing a biomass raw material to act on a supercritical water or subcritical water, in which by efficiently mixing the fluid containing a biomass raw material in a high concentration with the supercritical water or subcritical water, it is possible to allow the synthesis to proceed stably in a high yield while decreasing the amount of tar and carbon particles produced as by-products and suppressing blockage and abrasion of a pipe and an equipment. In addition, even in the case where by-products produced, such as tar, deposit onto the pipe wall surface, it is also an object of this invention to provide a method for easily removing the by-products.

In order to achieve the foregoing objects, a reaction apparatus of a supercritical water or subcritical water according to this invention includes a cylindrical mixing flow path for mixing at least one raw material fluid selected from the group consisting of glycerin, cellulose, and lignin with at least one of a supercritical water and a subcritical water; at least two inlet flow paths for flowing the raw material fluid and the supercritical water or subcritical water into the mixing flow path; an outlet flow path for discharging a reaction solution mixed in the mixing flow path; and an agitation blade having a rotating shaft set on a center shaft of the mixing flow path.

In addition, another reaction apparatus of a supercritical water or subcritical water according to this invention includes the above-described mixing flow path of the above-described reaction apparatus as a first mixing flow path; a second mixing flow path for flowing the reaction solution discharged from the outlet flow path of the first mixing flow path and a cooling water thereinto; and an agitation blade having a rotating shaft set on a center shaft of the second mixing flow path.

In addition, a reaction method of a supercritical water or subcritical water according to this invention is a method for synthesizing at least one member selected from the group consisting of acrolein, glucose, and hydroxymethyl furfural by allowing a supercritical water or subcritical water to act on a raw material fluid containing at least one member selected from the group consisting of glycerin, cellulose, and lignin, which includes a step of synthesizing a reaction solution having the raw material fluid and the supercritical water or subcritical water mixed therein by rotation of an agitation blade within a cylindrical mixing flow path.

In addition, the reaction method of a supercritical water or subcritical water according to this invention includes subsequent to the above-described step of the reaction method, a step of mixing the reaction solution and a cooling water by rotation of an agitation blade within a second cylindrical mixing flow path.

According to this invention, since the raw material fluid containing at least one member of glycerin, cellulose, and lignin and the supercritical water or subcritical water are agitated rapidly and sufficiently by the agitation blade set within the mixing flow path, mixing properties between the raw material fluid and the supercritical water or subcritical water, both of which are largely different in the density from each other due to the high concentration of the raw material, can be enhanced. As a result, it becomes possible to decrease the amount of tar produced as a reaction by-product and to increase a fluid shear force on the wall surface of the mixing flow path. Thus, it is possible to exfoliate and remove carbon produced due to carbonization of the tar deposited onto the reaction pipe wall surface. Furthermore, the agitation blade is rotated by kinetic energy of the mixed fluid, so that an external rotation drive device is not necessary. Thus, the structure can be simplified.

In addition, since the reaction solution originating from the reaction in the first mixing pipe and the cooling water can be mixed rapidly and sufficiently by the agitation blade set within the second mixing pipe, mixing properties between the raw material fluid and the supercritical water or subcritical water, both of which are largely different in the density from each other due to the high concentration of the raw material, can be enhanced, and it is possible to decrease the amount of tar produced as a reaction by-product. In addition, a fluid shear force on the wall surface of the mixing flow path can be increased, and hence, it is possible to exfoliate and remove carbon produced due to carbonization of the tar deposited onto the reaction pipe wall surface. In addition, the agitation blade is rotated by kinetic energy of the mixed fluid, so that an external rotation drive device is not necessary. Thus, the structure can be simplified.

### BRIEF DESCRIPTION OF THE DRAWINGS

FIG. 1 shows a dehydration reaction route of glycerin using a supercritical water.

FIG. 2 shows a related-art technology disclosed in JP-A-9-299966.

FIG. 3 shows a related-art technology disclosed in JP-A-10-015566.

FIG. 4 shows a related-art technology disclosed in JP-A-11-114600.

FIG. 5 shows an entire route of a synthesis apparatus of acrolein according to this invention.

FIG. 6 shows a mixing pipe according to Example 1 of this invention.

FIG. 7 shows a side view of an agitation shaft and an agitation blade in the mixing pipe according to Example 1 of this invention.

FIG. 8 shows a front view of the agitation shaft and the agitation blade in the mixing pipe according to Example 1 of this invention.

FIG. 9 shows a mixing pipe according to Example 2 of this invention.

FIG. 10 shows a mixing pipe according to Example 3 of this invention.

FIG. 11 shows an apparatus composed of mixing pipes of two stages according to Example 4 of this invention.

FIG. 12 shows temperature and pressure ranges of each of a supercritical water and a subcritical water in this invention.

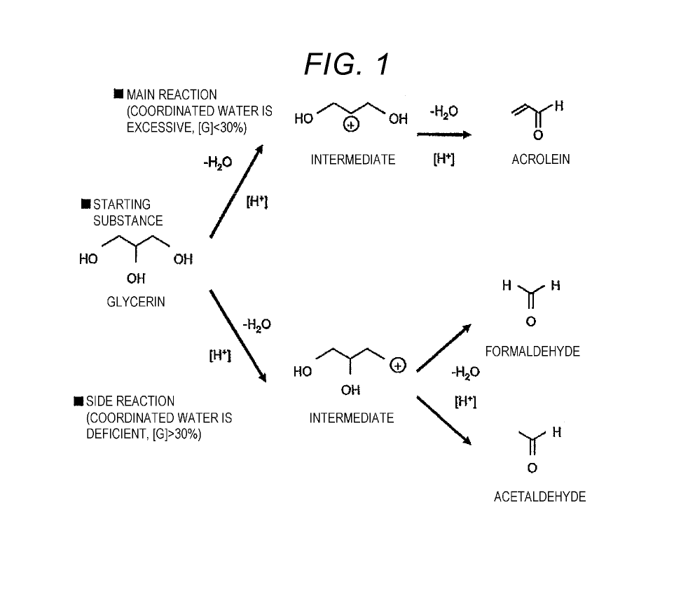

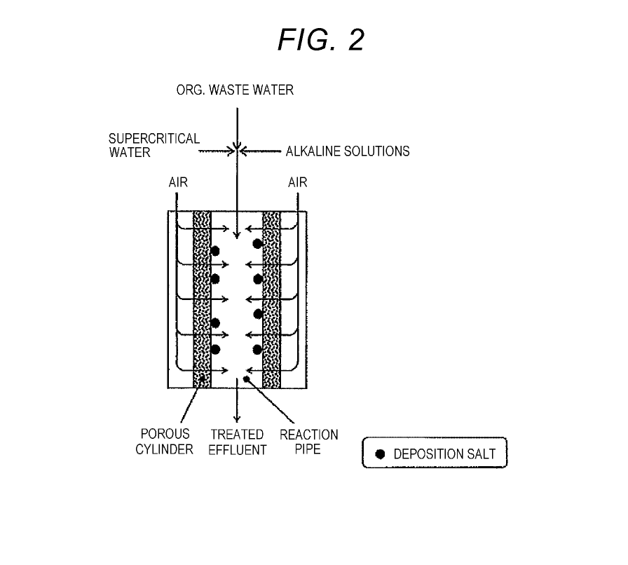

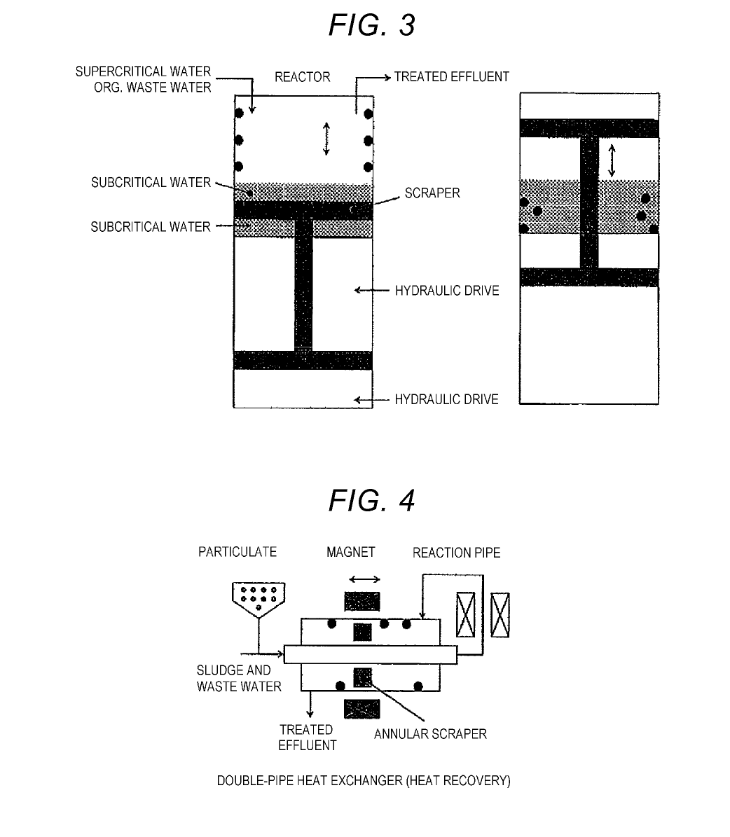

### DESCRIPTION OF EMBODIMENTS

A flow of selecting glycerin as a raw material and a supercritical water as water, mixing them to start a reaction, separating and removing by-products, and then recovering a reaction solution is described by reference to the accompanying drawings.

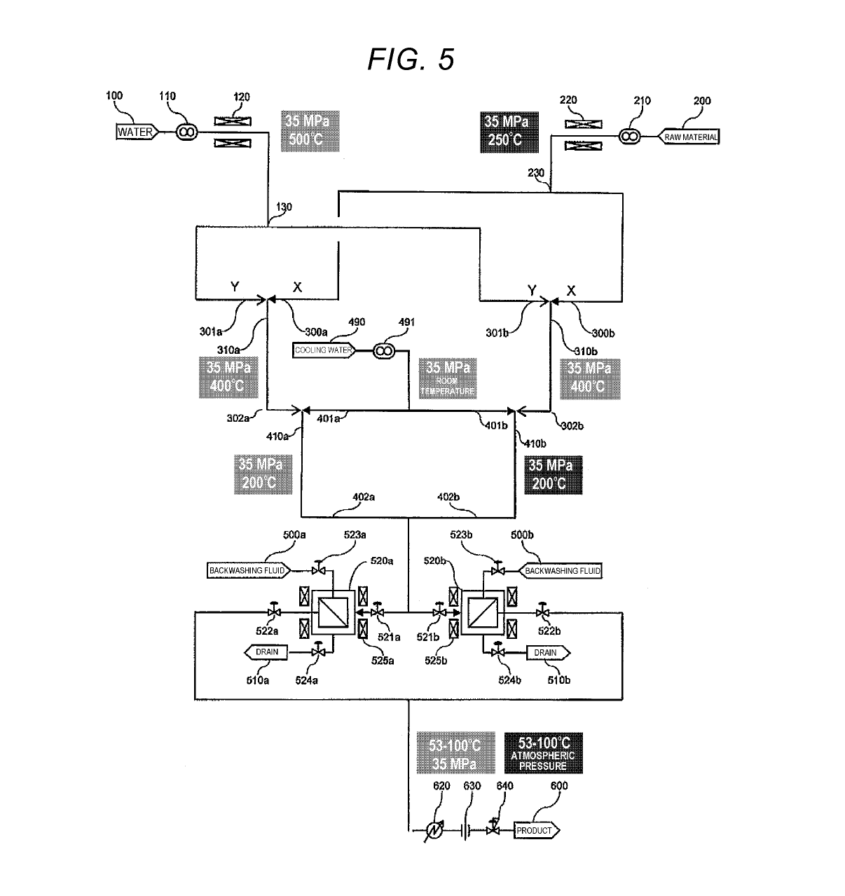

FIG. 5 shows an entire route of a synthesis apparatus of acrolein of this invention. First of all, water is sent at 35 MPa by a supercritical water high pressure pump (110), and the temperature is raised to 500° C. by a supercritical water pre-heater (120). In addition, a raw material composed of glycerin and dilute sulfuric acid is sent at 35 MPa by a raw material high pressure pump (210), and the temperature is raised to 250° C. by a raw material pre-heater (220). Such high-temperature water and high-temperature raw material are mixed in mixing pipes (310a and 310b) each having an agitator in a flow path, and an acrolein synthesis reaction is instantly started at 400° C. and 35 MPa.

### Example 1

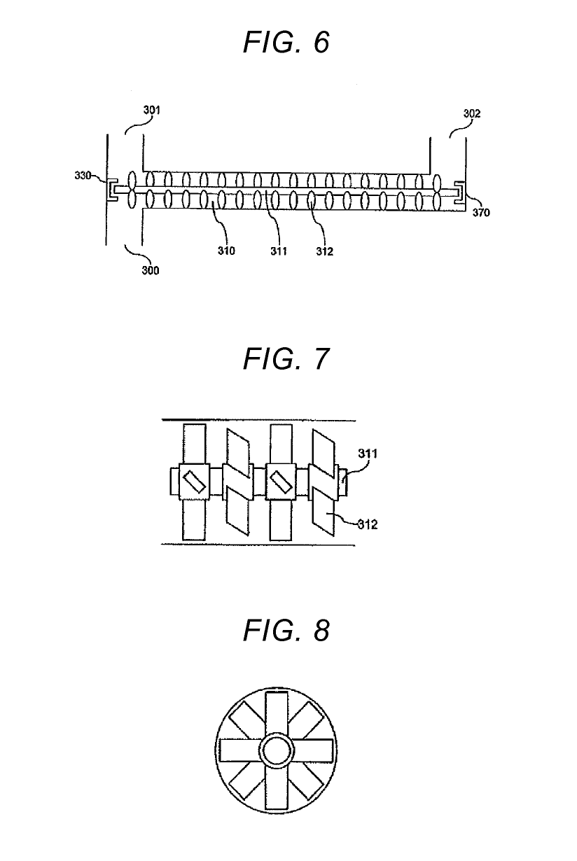

FIG. 6 shows a mixing pipe according to Example 1 of this invention and shows an example of a structure of a mixing flow path with a built-in agitator. A raw material fluid containing glycerin and sulfuric acid and a supercritical water are sent from an inlet flow path (300) and an inlet flow patch (301), respectively to a mixing pipe (310). An agitation blade (312) is connected to an agitation shaft (311) set within the mixing pipe (310) and rotated while allowing a center shaft of the mixing flow path to act as a rotating shaft by means of an upstream-side bearing (330) and a downstream-side bearing (370) of the mixing flow path, thereby performing mixing of the fluid.

The agitation blade is rotated by the kinetic energy of the fluid flowing through the mixing flow path. A diameter of the mixing flow path is from 1 to 10 cm, and a flow rate is desirably from 2 to 10 m/sec. This is because when the flow rate is less than 2 m/sec, the mixing performance is lowered, whereas when the flow rate is more than 10 m/sec, pipe wall thinning is caused due to erosion. In the case where the concentration of glycerin is 20 wt %, an optimum reaction time is from 1 to 2 seconds, and therefore, a length of a reaction pipe is from 4 to 20 m. For that reason, it is necessary to set the bearings (330) and (370) in two places of the upstream and downstream sides of the reaction pipe. It is desirable that a gap between the agitation blade and the agitation blade in the flow direction does not exist as far as possible. This is because by continuously setting the agitation blades in the flow direction, carbon deposited onto the pipe wall surface can be equally removed. FIGS. 7 and 8 are a side view and a front view, respectively of the agitation shaft and the agitation blade of Example 1. For the purpose of increasing a rotating force of the agitation blade, the agitation blade is set in a state such that its phase is out of phase of the subsequent-stage agitation blade.

In Example 1, since the agitation can be performed by the agitation blades set within the mixing flow path, the mixing properties between the raw material fluid and the supercritical water or subcritical water, both of which are largely different in the density from each other due to the high concentration of the raw material, can be enhanced. For that reason, the amount of tar produced as a reaction by-product can be decreased.

In addition, by increasing a fluid shear force in the vicinity of the wall surface of the mixing flow path, it is possible to exfoliate and remove carbon produced due to carbonization of the tar deposited onto the reaction pipe wall surface. Furthermore, the agitation blade is rotated by the kinetic energy of the mixed fluid, so that an external rotation drive device is not necessary. As a result, the structure can be simplified.

It is desirable that the mixing pipe (310), the agitation blade (312), and the agitation shaft (311) are subjected to mirror-processing. According to this, not only the deposition of tar onto the reaction pipe wall surface is decreased, but the fluid shear energy for removing the deposited tar and carbon produced due to carbonization of the tar is decreased. For that reason, blockage of the pipe can also be prevented.

In addition, it is desirable that not only a fluid containing oxygen is flown from the inlet flow path of the mixing pipe, or an outlet flow path in the case of backwashing, but the mixing pipe can be heated to 500° C. or higher by an external heating unit of the mixing pipe. This is because the carbon which has become unable to be removed by a shear force produced by the agitator is removed by means of burning, thereby preventing blockage of the pipe.

### Example 2

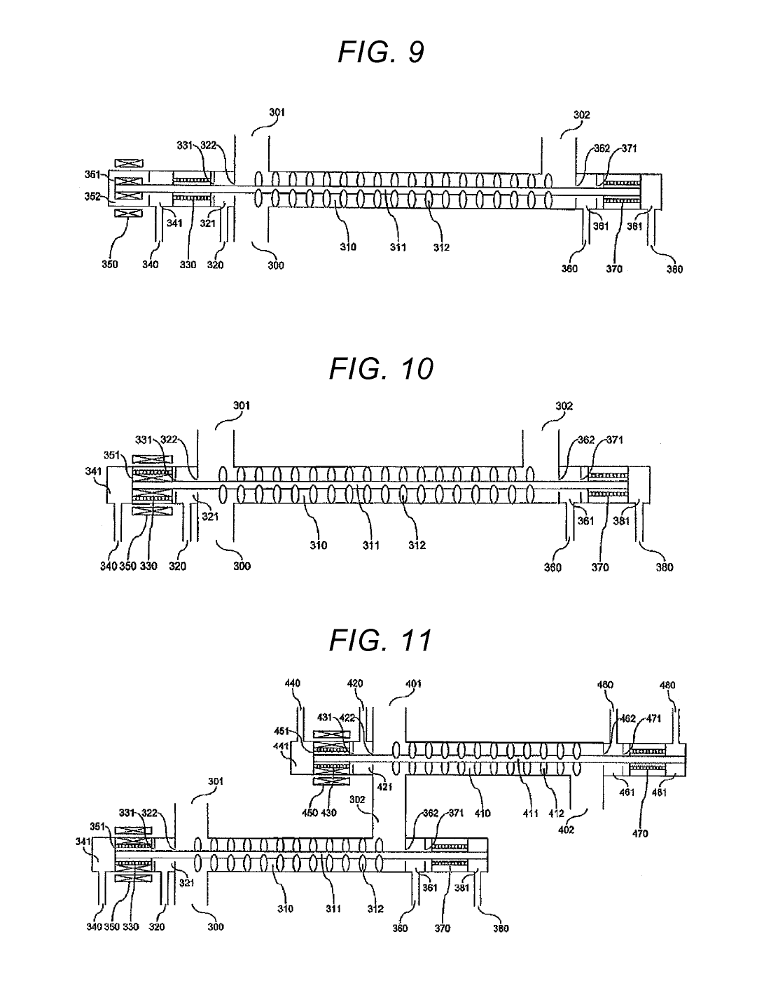

FIG. 9 shows a mixing pipe according to Example 2 of this invention and shows other example of a structure of a mixing flow path with a built-in agitator. In Example 2, the agitation blade is rotated by an external rotation drive system set outside the mixing pipe.

An upstream-side bearing (330) and a downstream-side bearing (370) of the mixing pipe are cooled by cooling waters flown from an upstream-side cooling water inlet (340) and a downstream-side cooling water inlet (380), respectively. In addition, an internal magnet (351) connected to a rotating shaft is cooled to not higher than the Curie point with a cooling water flown from the cooling water inlet (340).

In addition, it is necessary that a pressure of the cooling water is higher than a reaction pressure. According to this, the incorporation of a reaction solution into the cooling water is prevented, thereby avoiding a concern of deposition of by-products contained in the reaction solution, such as tar and carbon particles, onto the bearing or the magnet. In this way, by keeping the agitation strength high, the mixing properties are increased, thereby making it possible to decrease the amount of tar produced as a reaction by-product.

In addition, since a fluid shear force in the vicinity of the wall surface of the mixing flow path can be increased, it is possible to exfoliate and remove carbon produced due to carbonization of the tar deposited onto the reaction pipe wall surface. Furthermore, since the magnet can be kept at a temperature not higher than the Curie point by means of cooling, the agitation strength can be increased.

Incidentally, in the mixing flow path shown in FIG. 9, though an external magnet (350) and the internal magnet (351) are described on the upstream side of the mixing pipe, these magnets may also be provided on the downstream side. In the case where the magnets cannot be cooled, it is desirable to set these magnets on the downstream side. This is because the supercritical water of 500° C. flows into the upstream side, whereas the reaction temperature is relatively low as 400° C. on the downstream side.

In the case where a flow rate within the mixing pipe is defined as u (m/sec), and a width of the agitation blade in the flow direction is defined as w (m), it is desirable to set a rotation rate of the agitation blade to u/w (Hz). This is because the fluid always receives shearing by the agitation blade with its progress, and hence, the mixing properties can be enhanced.

### Example 3

FIG. 10 shows a mixing pipe according to Example 3 of this invention and shows other example of a structure of a mixing flow path with a built-in agitator. Example 3 is identical with Example 2 in the point that the agitation blade is rotated by an external rotation drive system set outside the mixing pipe. However, Example 3 is different from Example 2 in the point that the bearing is made of a magnet, and hence, the structure can be simplified.

In Examples 2 and 3 shown in FIGS. 9 and 10, after an optimum reaction time elapses within a mixing pipe (310), the cooling water is sent using a cooling water high pressure pump (491) shown in FIG. 5, and the reaction is stopped by means of direct mixing of the cooling water. On that occasion, in the case where the concentration of glycerin is 20%, since the optimum reaction time is from 1 to 2 seconds, it is necessary to subject the reaction solution to high-speed cooling to the reaction stopping temperature for a time of about 1/10 of the foregoing optimum reaction time. However, when the inner diameter of the reaction pipe is a size of several centimeters, from the standpoint of enhancing the controllability of the reaction time, it becomes necessary to adopt a direct mixing system of the cooling water rather than an indirect mixing system by a double-pipe cooler.

Next, an example in which by using a reaction apparatus using the above-described swirl flow in mixing the reaction solution and the cooling water, the controllability of the reaction time is enhanced, and the reaction yield is enhanced is shown.

### Example 4

FIG. 11 shows an apparatus according to Example 4 of this invention. Here, Example 4 is concerned with an example in which a mixing pipe apparatus having an agitation blade set within a flow path thereof is used in two stages including first mixing of a supercritical water and glycerin and second mixing of a reaction solution obtained by the first mixing and a cooling water.

By using the mixing pipe in two stages, since the reaction solution which has reacted in a first mixing pipe and the cooling water can be rapidly mixed by the agitation blade set within a second mixing pipe, the mixing properties between the raw material fluid and the supercritical water or subcritical water, both of which are largely different in the density from each other due to the high concentration of the raw material, can be enhanced, and it becomes possible to decrease the amount of tar produced as a reaction by-product. In addition, since a fluid shear force in the vicinity of the wall surface of the mixing flow path can be increased, it is possible to exfoliate and remove carbon produced due to carbonization of the tar deposited onto the reaction pipe wall surface.

In the reaction solution which has stopped the reaction, the tar and the carbon particles are separated by subsequent-stage filters (520a and 520b) shown in FIG. 5, and only the carbon particles are captured by the filters, whereas the tar is allowed to pass therethrough while keeping the high viscosity. According to this, blockage of the pipe due to coagulation of the tar and the carbon particles is prevented. Incidentally, when the operation time is prolonged, the backwashing efficiency is lowered, and a time interval of backwashing becomes short. In the case where the time interval of backwashing becomes short, by heating the filter to 500° C. or higher by a heater (525) set outside the filter and flowing a fluid containing oxygen to burn and remove the carbon particles, the backwashing performance can be recovered.

By preparing two or more systems for the filter for separating and removing of the carbon particles, a discharge work of the carbon particles (cake) deposited onto the filter by means of backwashing can be alternately performed. According to this, since it is not necessary to stop the whole of the plant, the continuous operation properties are enhanced, and a heat loss following starting of the plant can be decreased. Thus, it is possible to decrease the operation costs.

The reaction solution from which the carbon particles have been removed is cooled by a second cooler (620), the pressure of which is then decreased to atmospheric pressure by an orifice (630) and a pressure control valve (640), and the resulting reaction solution is sent to a subsequent-stage distillation apparatus of acrolein.

In each of the foregoing Examples, the case of synthesizing acrolein using glycerin and dilute sulfuric acid as the raw material and a supercritical water as water has been described. However, it should not be construed that this invention is limited to this case. The raw material may be glycerin, cellulose, or lignin, or a combination thereof, and a subcritical water may be used in place of the supercritical water. Then, the desired substance may be a material to be synthesized, which is at least one member of acrolein, glucose, and hydroxymethyl furfural.

### Example 5

An example of synthesizing glucose through a hydrolysis reaction of cellulose using a supercritical water or subcritical water is described by reference to FIG. 11. A cellulose slurry having cellulose dispersed in water is used for a raw material liquid, and a supercritical water is used as a reaction solvent. From 1 to 10% of a cellulose slurry aqueous solution is sent at from 20 to 40 MPa to a raw material fluid inlet (300). A supercritical water of the same pressure at from 400 to 600° C. is sent to a supercritical water or subcritical water inlet (301). The both are mixed at a high speed within a first mixing pipe (310) by an agitation blade (312) to achieve a hydrolysis reaction at from 20 to 40 MPa and from 200 to 400° C. for from 0.1 to 20 seconds. According to this hydrolysis reaction, a sugar such as glucose and fructose is synthesized. Thereafter, this reaction solution is rapidly mixed with a cooling water sent from a cooling water inlet (401) by an agitation blade set within a second mixing pipe and then cooled to 100° C. to 200° C., thereby stopping the reaction. Thereafter, the carbon particles are removed by a filter, and the reaction solution which has been cooled and decreased in the pressure is recovered.

### Example 6

An example of synthesizing 5-hydroxymethyl furfural through a dehydration reaction of lignin using a supercritical water or subcritical water is described by reference to FIG. 11. A solution obtained by adding 2 mM of sulfuric acid to a 20% lignin aqueous solution is used as a raw material liquid, and a supercritical water is used as a reaction solvent. The aqueous solution of lignin and the acid is sent to a raw material fluid inlet (300) at from 5 to 20 MPa and from 100 to 200° C. A supercritical water of the same pressure at from 200 to 400° C. is sent to a supercritical water or subcritical water inlet (301). The both are mixed at a high speed within a first mixing pipe (310) by an agitation blade (312) to achieve a dehydration reaction at from 5 to 20 MPa and from 200 to 350° C. for from 5 to 30 seconds. According to this dehydration reaction, 5-hydroxymethyl furfural is synthesized. Thereafter, this reaction solution is rapidly mixed with a cooling water sent from a cooling water inlet (401) by an agitation blade set within a second mixing pipe and then cooled to 100° C. to 200° C., thereby stopping the reaction. Thereafter, the carbon particles are removed by a filter, and the reaction solution which has been cooled and decreased in the pressure is recovered.

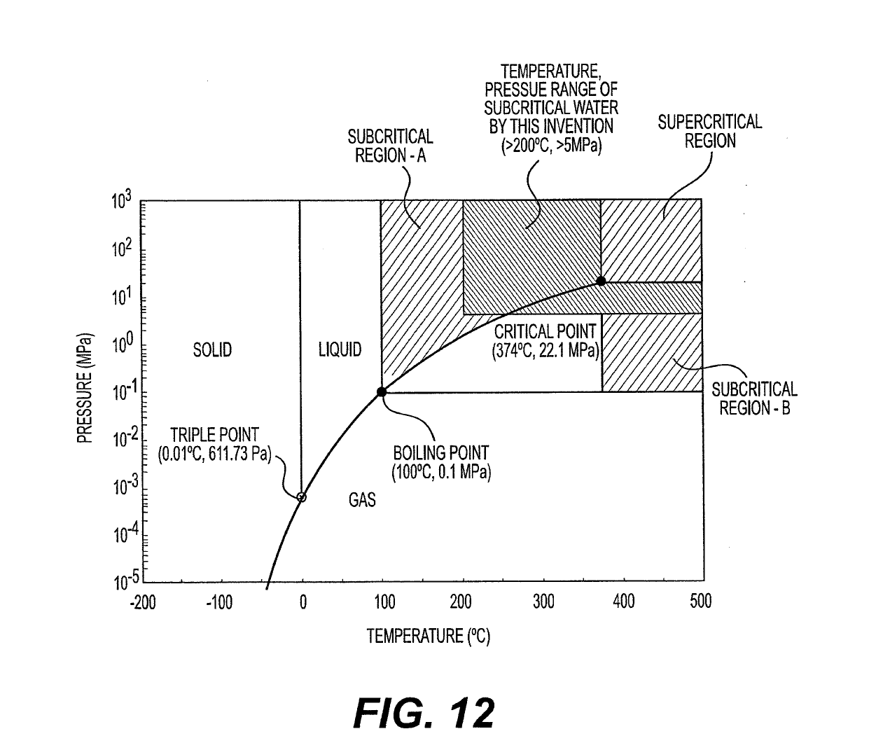

In this invention, the supercritical water is defined to be water in a state at 374° C. or higher and 22.1 MPa or more. In addition, the subcritical water includes plural definitions. The subcritical water is frequently defined so as to include water of from 100° C. to 374° C. and a saturated vapor pressure or more (subcritical region A) and water of from 374° C. or higher and from 0.1 to 22.1 MPa (subcritical region B). As shown in FIG. 12, temperature and pressure ranges of the subcritical water in this invention are defined to be those of from 200 to 374° C. and from 5.0 to 22.1 MPa.

In addition, in this invention, though the biomass is not limited, it refers to an oil or fat, a woody biomass, a rice straw, cellulose, a wastepaper, a sugar, glucose, or fructose.

What is claimed is:

1. A reaction apparatus of a supercritical water or subcritical water comprising
a cylindrical mixing flow path for mixing at least one raw material fluid selected from the group consisting of glycerin, cellulose, and lignin with at least one of a supercritical water and a subcritical water;
at least two inlet flow paths for flowing the raw material fluid and the supercritical water or subcritical water into the mixing flow path;
an outlet flow path for discharging a reaction solution mixed in the mixing flow path; and
an agitation blade having a rotating shaft set on a center shaft of the mixing flow path.

2. The reaction apparatus of a supercritical water or subcritical water according to claim 1, wherein
the rotating shaft is rotated by kinetic energy of the reaction solution.

3. The reaction apparatus of a supercritical water or subcritical water according to claim 1, wherein
a magnet and a bearing fixed to the rotating shaft are provided;
a magnet for rotating the magnet in a non-contact state by a magnetic force is provided outside the reaction apparatus; and
an inlet flow path for flowing a cooling water therein at a pressure higher than a pressure of the mixing flow path and an outlet flow path are provided in a vessel having the magnet and the bearing fixed to the rotating shaft housed therein.

4. The reaction apparatus of a supercritical water or subcritical water according to claim 1, wherein
a liquid contact part within the reaction apparatus, which comes into contact with the reaction solution, is subjected to mirror-processing.

5. A reaction apparatus of a supercritical water or subcritical water comprising
the mixing flow path of the reaction apparatus according to claim 1 as a first mixing flow path;
a second mixing flow path for flowing the reaction solution discharged from the outlet flow path of the first mixing flow path and a cooling water thereinto; and
an agitation blade having a rotating shaft set on a center shaft of the second mixing flow path.

6. The reaction apparatus of a supercritical water or subcritical water according to claim 5,
comprising, subsequent to the second mixing flow path, a filter for separating tar and carbon particles;
a heating unit capable of heating the filter to 500° C. or higher; and
an equipment for flowing a fluid containing oxygen in the mixing flow path,
wherein a function to burn and remove the carbon particles deposited onto the filter is provided.

7. A reaction method of a supercritical water or subcritical water for synthesizing at least one member selected from the group consisting of acrolein, glucose, and hydroxymethyl furfural by allowing a supercritical water or subcritical water on a raw material fluid containing at least one member selected from the group consisting of glycerin, cellulose, and lignin, comprising
a step of synthesizing a reaction solution having the raw material fluid and the supercritical water or subcritical water mixed therein by rotation of an agitation blade within a cylindrical mixing flow path.

8. The reaction method of a supercritical water or subcritical water according to claim 7, wherein
the agitation blade having the rotating shaft is rotated by kinetic energy of the reaction solution.

9. The reaction method of a supercritical water or subcritical water according to claim 7, wherein
the rotation of the agitation blade having the rotating shaft is performed by a magnetic force of a magnet provided outside the flow path in a non-contact state with a magnet fixed to the rotating shaft, and a bearing and the magnet fixed to the rotating shaft are cooled with a cooling water having a pressure higher than a reaction pressure.

10. The reaction method of a supercritical water or subcritical water according to claim 7,
comprising, subsequent to the step of the reaction method, a step of mixing the reaction solution and a cooling water by rotation of an agitation blade within a second cylindrical mixing flow path.

11. The reaction method of a supercritical water or subcritical water according to claim 10, wherein
at the time of backwashing of a filter for separating tar and carbon particles from the reaction solution discharged from the second mixing flow path, the filter is heated to 500° C. or more, a fluid containing oxygen is flown into the mixing flow path, and the carbon particles deposited onto the filter are burnt and removed.
